(19) United States
(12) Patent Application Publication
(10) Pub. No.: US 2019/0224331 A1
(43) Pub. Date: Jul. 25, 2019

(54) METABOLIC DRUG LOADING OF EVs

(71) Applicant: Evox Therapeutics Ltd, Oxford (GB)

(72) Inventor: Oscar WIKLANDER, Solna (SE)

(21) Appl. No.: 16/317,432

(22) PCT Filed: Jul. 10, 2017
(86) PCT No.: PCT/EP2017/067237

§ 371 Date: Jan. 11, 2019

(30) Foreign Application Priority Data
Jul. 11, 2016 (GB) 1611990.1

(51) Int. Cl. A61K 47/66; A61K 35/13; A61K 35/545; A61P 35/00; A61K 47/54; A61K 47/69; A61K 35/28

(52) U.S. Cl. CPC A61K 47/66; A61K 35/13; A61K 35/545; A61K 47/6943; A61K 47/544; A61K 47/6901; A61K 35/28; A61P 35/00

(57) ABSTRACT

The present invention relates to methods for metabolically loading extracellular vesicles (EVs) with a pharmacological agent. The methods comprise culturing EV source cells in the presence of a metabolic component comprising a pharmacological agent, which is thereby incorporated into the EV-producing cells and subsequently into the EV. The invention further relates to medical uses and compositions of such EVs.

### TECHNICAL FIELD

The present invention relates to methods for loading extracellular vesicles (EVs) with pharmacological agents and uses of such EVs for therapeutic purposes.

### BACKGROUND ART

Extracellular vesicles (EVs) modulate cell-to-cell communication in normal physiology and pathology by presenting their contents (primarily RNA, proteins, and lipids) to recipient cells in target tissues. Modification of EVs to incorporate various types of pharmacological agents have been explored in numerous contexts, for instance WO2013/084000, which discloses the use of exosomes for intracellular delivery of biotherapeutics, or WO2010/119256, which describes delivery of exogenous genetic material using exosomes.

The utility of EVs as drug delivery vehicles is unquestionable in the case of for instance nucleic acid based drugs such as siRNA, large protein-based drugs targeting intracellular components, and e.g. poorly soluble or highly toxic pharmacological agents. EV-mediated small molecule drug delivery has also been explored to a great extent, with for instance WO2011/097480 representing the typical approach to loading of EVs with organic small molecule compounds. WO2011/097480 describes a very facile method wherein e.g. the phytochemical small molecule agents curcumin and resveratrol are loaded into EVs using a simple co-incubation step during which purified EVs and free drug (e.g. curcumin) are allowed to incubate together in phosphate buffered saline (PBS) at room temperature, relying on diffusion of the drug into the EV. Although highly convenient and straightforward, this conventional approach to loading small molecule agents into EVs is not particularly efficient, results in significant waste of the small molecule, and is also very difficult to control. Others (for instance Fuhrman et al, J. Control Rel., 2015) have also evaluated permeabilization of EVs, using detergents such as saponin, as a way of increasing the loading efficiency of, in this case, the photoactive small molecule agent porphyrin.

A recent patent application (WO2015/120150) is also concerned with loading of tumor-derived EVs with various types of anticancer drugs, mentioning both small molecules and large biopharmaceuticals. However, as is often the case in the art, very little information is available on how to load exosomes and if there are methods available they are rarely useful for loading and actual therapeutic application of EVs carrying pharmacological agents.

### SUMMARY OF THE INVENTION

It is hence an object of the present invention to overcome the above-identified problems associated with the loading of pharmacological agents into EVs for subsequent therapeutic, prophylactic and/or diagnostic application. Furthermore, the present invention aims to satisfy other existing needs within the art, for instance to enable loading of significant amounts of pharmacological agents into EVs, to enable controllable loading, and to provide pharmacological agent-loaded EVs with considerable therapeutic potential. Furthermore, the present invention provides for a generally applicable strategy for loading of pharmacological agents of various origins (non-limiting examples of pharmacological agents encompassed within the present invention include small organic compounds, RNA therapeutics, peptides and proteins, etc.) into EVs, which has been lacking in the art.

The present invention achieves these and other objectives by harnessing metabolic pathways for loading of EVs. Thus, in one aspect, the present invention relates to methods for metabolically loading EVs with a pharmacological agent, comprising the step of culturing EV source cells in the presence of a conjugate comprising a pharmacological agent conjugated to a metabolic component. Naturally, the EVs produced by the EV source cells are typically subsequently be harvested, typically from the cell culture media into which they may be released, and purified for further use.

In another aspect, the present invention pertains to an EV comprising a pharmacological agent conjugated to a metabolic component. Suitable metabolic components may include lipids such as phospholipid, peptides, sterols such as cholesterol, vitamins such as vitamin B12, etc. In advantageous embodiments the conjugation between the metabolic component and the pharmacological agent to be carried by the EV may be designed to be releasable and/or cleavable, in order to release the pharmacological agent in the EV and potentially subsequently into a target location. Thus, in one aspect, the present invention also relates to EVs comprising a pharmacological agent of interest, wherein the drug has been released in the EV from a conjugate comprising a metabolic component.

The term “pharmacological agent” or “drug” as used herein encompasses a wide variety of different types of molecules, including peptides, nucleic acid-based agents such as siRNA or mRNA, and organic compounds. More in detail, the pharmacological agents of the present invention may be selected from a wide variety of drug or diagnostic agent categories, for instance anticancer agents such as doxorubicin, methotrexate, 5-fluorouracil or other nucleoside analogues such as cytosine arabinoside, proteasome inhibitors such as bortezomib, or kinase inhibitors such as imatinib or seliciclib, or NSAIDs such as naproxen, aspirin, or celecoxib, antibiotics such as heracillin, antihypertensives such as ACE inhibitors such as enalapril, short interfering RNAs for silencing of various target genes, mRNA and modified mRNAs to enable delivery of a protein-coding agent, splice-switching RNAs to change splicing patterns, peptides with pharmacological activity or endosomal escape activity, protein therapeutics, etc.

In yet another aspect, the present invention pertains to EV-based methods for delivering pharmacological agents to a target location, such as a target cell, a target tissue, a target organ, or to any target compartment (which may also include bodily fluids, for instance the blood stream or cerebrospinal fluid). Such methods comprise exposing the target location to EVs loaded with a pharmacological agent, either in the form of a conjugate with a metabolic component or a pharmacological agent that has been released from a conjugate in the EV.

In a further aspect, the present invention also relates to methods of altering the pharmacokinetic or pharmacodynamics profile of a pharmacological agent. Such methods comprise metabolically loading the pharmacological agent in question into an EV, in order to modulate in vivo and potentially also in vitro properties of the agent.

Additionally, in further aspect, the present invention pertains to pharmaceutical compositions comprising EVs carrying pharmacological agents as per the present invention, or in practical terms compositions comprising populations of EVs comprising pharmacological agents as per the present invention. The EV concentration in such compositions may be expressed in many different ways, for instance amount of EV protein per unit (often volume) or per dose, number of particles per unit (often volume) or per dose, concentration of small molecule drug per unit or per dose, etc. Typically, such pharmaceutical compositions are formulated for in use in vivo and also in vitro using pharmaceutically acceptable excipients.

Finally, the present invention also relates to medical uses and applications of EVs comprising different types of pharmacological agents, for instance in the treatment of inflammatory diseases, autoimmune diseases, cancer, metabolic disorders, central nervous system diseases, neuromuscular diseases, or any suitable disease or disorder.

### BRIEF DESCRIPTION OF THE DRAWINGS

FIG. 1 shows the antitumor effects of PBMC-derived EVs containing C18 fatty acid-conjugated cytosine arabinoside (C18-AraC), assayed in MDA-MB-231 cells using an MTT assay.

FIG. 2 shows COX-2 inhibition of EVs comprising phospholipids conjugated to isobutylphenylpropanoic acid, as measured in a RAW264.7 cell model.

FIG. 3 shows COX-2 inhibition of EVs loaded with IBU-vitamin B7 and IBU-vitamin B9, measured in RAW264.7 cells.

### DETAILED DESCRIPTION OF THE INVENTION

The present invention describes inter alia novel methods, compositions, EVs, and uses of EVs for the delivery of pharmacological agents. More specifically, the present invention relates to methods for EV loading, EVs loaded with pharmacological agents, various methods for utilizing such EVs, pharmaceutical compositions comprising EVs in therapeutically effective amounts, and medical uses of drug-loaded EVs as per the present invention.

For convenience and clarity, certain terms employed herein are collected and described below. Unless otherwise defined, all technical and scientific terms used herein have the same meaning as commonly understood by one of ordinary skill in the art to which this invention belongs.

Where features, aspects, embodiments, or alternatives of the present invention are described in terms of Markush groups, a person skilled in the art will recognize that the invention is also thereby described in terms of any individual member or subgroup of members of the Markush group. The person skilled in the art will further recognize that the invention is also thereby described in terms of any combination of individual members or subgroups of members of Markush groups. Additionally, it should be noted that embodiments and features described in connection with one of the aspects and/or embodiments of the present invention also apply mutatis mutandis to all the other aspects and/or embodiments of the invention. For example, the various small molecules described in connection with the methods for metabolically based loading of EVs are to be understood to be disclosed, relevant and included also in the context of the pharmaceutical compositions comprising EVs. Furthermore, certain embodiments described in connection with certain aspects, for instance the administration routes of the drug-loaded EVs, as described in relation to aspects pertaining to treating certain medical indications with EVs as such, may naturally also be relevant in connection with other aspects and/or embodiment such as those pertaining to the pharmaceutical compositions of the present invention. Moreover, any and all features (for instance any and all members of a Markush group) can be freely combined with any and all other features (for instance any and all members of any other Markush group), e.g. any metabolic component may be combined with any pharmacological agent, or any EV cell source may be combined with any EV protein which in turn may be combined with any targeting agent. Furthermore, when teachings herein refer to EVs in singular and/or to EVs as discrete natural nanoparticle-like vesicles it should be understood that all such teachings are equally relevant for and applicable to a plurality of EVs and populations of EVs. As a general remark, the pharmacological agents, the metabolic components, the targeting moieties, the cell sources, the exosomal proteins, and all other aspects, embodiments, and alternatives in accordance with the present invention may be freely combined in any and all possible combinations without deviating from the scope and the gist of the invention. Furthermore, any polypeptide or polynucleotide or any polypeptide or polynucleotide sequences (amino acid sequences or nucleotide sequences, respectively) of the present invention may deviate considerably from the original polypeptides, polynucleotides and sequences as long as any given molecule retains the ability to carry out the technical effect associated therewith. As long as their biological properties are retained the polypeptide and/or polynucleotide sequences according to the present application may deviate with as much as 50% (calculated using for instance BLAST or ClustalW) as compared to the native sequence, although a sequence identity that is as high as possible is preferable (for instance 60%, 70%, 80%, or e.g. 90% or higher). The combination (fusion) of e.g. at least one targeting polypeptide and at least one exosomal protein implies that certain segments of the respective polypeptides may be replaced and/or modified, meaning that the deviation from the native sequence may be considerable as long as the key properties (e.g. the targeting properties and trafficking to the surface of exosomes in this particular case) are conserved. Similar reasoning thus naturally applies to the polynucleotide sequences encoding for such polypeptides.

The terms “extracellular vesicle” or “EV” or “exosome” are used interchangeably herein and shall be understood to relate to any type of vesicle that is obtainable from a cell in any form, for instance a microvesicle (e.g. any vesicle shed from the plasma membrane of a cell), an exosome (e.g. any vesicle derived from the endo-lysosomal pathway), an apoptotic body (e.g. obtainable from apoptotic cells), a microparticle (which may be derived from e.g. platelets), an ectosome (derivable from e.g. neutrophils and monocytes in serum), prostatosome (e.g. obtainable from prostate cancer cells), or a cardiosome (e.g. derivable from cardiac cells), etc. Furthermore, the said terms shall also be understood to relate to extracellular vesicle mimics, cell and/or cell membrane-based vesicles obtained through cell extrusion, membrane extrusion, vesicle extrusion, or other techniques, etc. Essentially, the present invention may relate to any type of lipid-based structure (with vesicular morphology or with any other type of suitable morphology) that can act as a delivery or transport vehicle for pharmacological agents of interest. It will be clear to the skilled artisan that when describing medical and scientific uses and applications of the EVs, the present invention normally relates to a plurality of EVs, i.e. a population of EVs which may comprise thousands, millions, billions or even trillions of EVs. As can be seen from the experimental section below, EVs may be present in concentrations such as 10⁵, 10⁸, 10¹¹, 10¹⁵, 10¹⁸, 10²⁵, 10³⁰ EVs (often termed “particles”) per unit of volume (for instance per ml), or any other number larger, smaller or anywhere in between. In the same vein, the term “population”, which may e.g. relate to an EV comprising a certain pharmacological agent and often a certain metabolic component, shall be understood to encompass a plurality of entities constituting such a population. In other words, individual EVs when present in a plurality constitute an EV population. Thus, naturally, the present invention pertains both to individual EVs comprising pharmacological agents and populations comprising EVs comprising pharmacological agents, as will be clear to the skilled person. The dosages of EVs when applied in vivo may naturally vary considerably depending on the disease to be treated, the administration route, the pharmacological agent cargo, etc.

The term “pharmacological agent” or “pharmacologically active agent” or “therapeutic agent” or “drug” or “pharmacological drug” or “pharmacological therapeutic” are used interchangeably herein and shall be understood to relate to any molecular agent which may be used for the treatment and/or prophylaxis and/or diagnosis of a disease and/or disorder. Pharmacological agents as per the present invention encompass a wide variety of pharmacologically or pharmaceutically active agents, including (i) organic compounds with pharmacological activity which are normally synthesized via chemical synthesis, (ii) naturally derived compounds which may for instance be obtained via purification from natural sources, (iii) nucleic acid-based compounds of various kinds, for instance oligonucleotides such as siRNA, splice-switching RNA, CRISPR guide strands, short hairpin RNA (shRNA), antisense oligonucleotides, polynucleotides such as mRNA, and in particular nucleic acid-based agents which are chemically synthesized and/or which comprise chemically modified nucleotides such as 2′-O-Me, 2′-O-Allyl, 2′-O-MOE, 2′-F, 2′-CE, 2′-EA 2′-FANA, LNA, CLNA, ENA, PNA, phosphorothioates, tricyclo-DNA, etc., (iii) peptides and polypeptides (i.e. proteins) of any kind, for instance obtained via peptides synthesis or via recombinant protein production, and (iv) essentially any type of pharmacological and/or pharmaceutically active agent which can conjugated to a suitable metabolic component. The present invention is naturally applicable also to other pharmacological agents without departing from the gist of the invention, as would be clear to a person skilled in the art.

The terms “metabolic component” or “metabolic molecule” are used interchangeably herein and shall be understood to relate to any molecule that can be utilized by a cell or a derivative of a cell (such as an EV) for essentially any purpose, be it as a structural component (such as a phospholipid or a cholesteryl moiety), an intermediary in an anabolic or catabolic pathway (such as pyruvate), a co-factor for an enzyme (such as vitamin B12), or any other type of molecule that can be used, incorporated and/or in any way metabolized or included in a cell, normally an EV source cell. Non-limiting examples as per the present invention include lipids, fatty acids, mono-, di- or polysaccharides, vitamins, sterols, ganglioside, peptides, proteins, nucleosides and nucleotide, and any combinations thereof. Non-limiting examples of metabolic components include fatty acids comprising e.g. 4-30 carbon such as stearic acid, lauric acid, myristic acid, palmitic acid, arachidic acid, and behenic acid, or any derivatives thereof, especially un-saturated fatty acid derivatives thereof. Other non-limiting examples of lipids include phospholipids, sphingolipids, glycolipids, cholesterol and other sterols, mono-, di- or tri-glycerides. Additional non-limiting examples include vitamins A, B, C, D, E, K, and all vitamers thereof (for instance B7 (biotin), B9 (folate), B12, etc.), sugars and sugar analogues, nucleotides and nucleosides and analogues thereof, amino acids and their analogues, etc.

The terms “EV protein”, “exosomal protein”, “exosomal sorting domain”, “EV sorting domain”, “EV sorting protein”, “exosomal protein”, “exosomal polypeptide”, “EV polypeptide”, etc. are used interchangeably herein and shall be understood to relate to any polypeptide that can be utilized to transport a polypeptide construct (which typically comprises, in addition to the EV protein, at least one protein of interest) to a suitable vesicular structure, i.e. to a suitable EV. More specifically, said terms shall be understood as comprising any polypeptide that enables transporting, trafficking or shuttling of a polypeptide construct to a vesicular structure, such as an exosome. Examples of such exosomal sorting domains are for instance CD9, CD53, CD63, CD81, CD54, CD50, FLOT1, FLOT2, CD49d, CD71, CD133, CD138, CD235a, ALIX, Syntenin-1, Syntenin-2, Lamp2b, TSPAN8, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11b, CD11c, CD18/ITGB2, CD41, CD49b, CD49c, CD49e, CD51, CD61, CD104, Fc receptors, interleukin receptors, immunoglobulins, MHC-I or MHC-II components, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD274, CD362, COL6A1, AGRN, EGFR, GAPDH, GLUR2, GLUR3, HLA-DM, HSPG2, L1CAM, LAMB1, LAMC1, LFA-1, LGALS3BP, Mac-1 alpha, Mac-1 beta, MFGE8, SLIT2, STX3, TCRA, TCRB, TCRD, TCRG, VTI1A, VTI1B, and any combinations thereof, but numerous other polypeptides capable of transporting a polypeptide construct to an EV are comprised within the scope of the present invention. The EV proteins as per the present invention are typically of human origin and can be found in various publicly available databases such as Uniprot, RCSB, etc. The EV proteins may be fused to various other proteins and/or protein domains, to for instance enhance the surface display, increase avidity, or enable interaction with particular types of binding proteins.

The terms “source cell” or “EV source cell” or “parental cell” or “cell source” or “EV-producing cell” or any other similar terminology shall be understood to relate to any type of cell that can produce EVs under suitable conditions, for instance in suspension culture or in adherent culture or any in other type of culturing system. Source cells as per the present invention may also include cells producing exosomes in vivo. The source cells per the present invention may be select from a wide range of cells and cell lines, for instance mesenchymal stem or stromal cells or fibroblasts (obtainable from e.g. bone marrow, adipose tissue, Wharton's jelly, perinatal tissue, amniotic tissues and/or fluid, tooth buds, umbilical cord blood, skin tissue, etc.), amnion cells and more specifically amnion epithelial cells optionally expressing various early markers, myeloid suppressor cells, M2 polarized macrophages, adipocytes, endothelial cells, fibroblasts, etc. Cell lines of particular interest include human umbilical cord endothelial cells (HUVECs), human embryonic kidney (HEK) cells, endothelial cell lines such as microvascular or lymphatic endothelial cells, chondrocytes, MSCs of different origin, airway or alveolar epithelial cells, fibroblasts, endothelial cells, etc. Also, immune cells such as B cells, T cells, NK cells, macrophages, monocytes, dendritic cells (DCs) are also within the scope of the present invention, and essentially any type of cell which is capable of producing EVs is also encompassed herein.

In a first aspect, the present invention relates to methods for metabolically loading EVs with pharmacological agents, wherein the methods comprise culturing EV source cells in the presence of a conjugate comprising a pharmacological agent conjugated to a metabolic component. More specifically, in one embodiment, EV source cells are exposed to a conjugate comprising a pharmacological agent and a metabolic component which via essentially any mechanism is incorporated into the source cells and naturally into the EVs obtained from the EV source cell. The conjugate typically comprises a chemical link between the metabolic component and the pharmacological agent, and this chemical linkage may optionally dissociate, break, release, or get cleaved to release the pharmacological agent. Although advantageous, release of the pharmacological agent from the metabolic component is not a requirement, as long as the pharmacological agent can exert its pharmacological effect while bound to the metabolic component. Suitable non-limiting examples of releasable chemical linkages are disulfide bridges or thioether bonds which may become undergo reduction in reductive environments, amide bonds which may be cleaved by e.g. proteases and other enzymes, biotin-streptavidin linkages which dissociate under certain in vivo conditions, etc. There are nonetheless multiple strategies available for covalent conjugation/linkage of a pharmacological agent to a metabolic component, with non-limiting examples such as an ester bond, an amide bond, a disulfide bond, a thioether bond, a biotin-streptavidin interaction, a linkage obtained through a maleimide-NHS reaction, a linkage obtained through a EDC-NHS reaction, a stapled linkage (for instance an all-hydrocarbon staple) and various other bonds.

In further embodiments, the EV source cells may be cultured under cell culture conditions which favor metabolic incorporation of the metabolic component. Suitable examples of such conditions would be to use a cell culture media with low concentration or essentially complete absence of e.g. vitamin B12 or biotin, naturally except for vitamin B12 or biotin conjugated to the pharmacological agent which is to be metabolically loaded into the EVs. The low concentration/absence of the metabolic component drives the metabolic uptake, processing and incorporation, thereby increasing the loading into the EV source cell and into the EVs. Alternative ways of favoring metabolic incorporation are culturing EV source cells in under low oxygen (hypoxic conditions), cytokine exposure, and/or other forms of cellular stress, for instance exposure to agents such as bafilomycin.

In another alternative aspect, the present invention relates to methods of metabolically loading pharmacological agents into EVs by exposing EVs directly (and not EV source cells) to conjugates comprising a pharmacological agent and a metabolic component, to enable direct incorporation into the EV as such. Non-limiting examples of such methods include loading based on conjugating a pharmacological agent to e.g. lipids such as a sphingolipid, ceramide, cholesterol, a phospholipid or a fatty acid, or a ganglioside such as GM1, or a sterol and/or a peptide or a protein (such as a conventional EV protein or a peptide/protein which interacts with an EV protein and hence an EV), or any other type of suitable metabolic component. Furthermore, the use of electroporation and/or mixing of the conjugate with e.g. a transfection reagent are also within the scope of the present invention. The loading of pharmacological agents into EVs and/or into parental cells can increase when electroporation and/or transfection reagents (e.g. liposomes, cell-penetrating peptides, cationic polymers such as PEI, lipid nanoparticles, etc.) are employed. Electroporation may be carried out using voltages in the range of 20 V/cm to 1000 V/cm, often 20 V/cm to 100 V/cm. The capacitance of the electroporation step is normally between 25 μF and 250 μF, such as between 25 μF and 125 μF, although these parameters may vary extensively depending on various factors such as the EV source cells, any genetic or chemical modification of the EV, the nature of the metabolic component, the nature of the pharmacological agent, etc.

In another embodiment, the present invention relates to an EV comprising a pharmacological agent conjugated to a metabolic component. The pharmacological agent is typically conjugated to the metabolic component via a chemical link, which may optionally be capable of releasing the pharmacological agent. The release of the pharmacological agent may advantageously result in location of free small molecule drug e.g. inside the EV or into the EV membrane. The way chemical link may be released as a result of e.g. enzymatic cleavage, dissociation, lysis, or any other type of breaking of the chemical link.

As abovementioned, the pharmacological agents as per the present invention can be obtained from essentially the entire space of pharmaceutically and/or pharmacologically and/or diagnostically relevant agents, for instance anticancer agents, cytostatic agents, tyrosine kinase inhibitors, statins, NSAIDs, antibiotics, antifungal agents, antibacterial agents, anti-inflammatory agents, anti-fibrotics, antihypertensives, aromatase or esterase inhibitors, an anticholinergics, SSRIs, BKT inhibitors, PPAR agonists, HER inhibitors, AKT inhibitors, BCR-ABL inhibitors, signal transduction inhibitors, angiogenesis inhibitors, synthase inhibitors, ALK inhibitors, BRAF inhibitors, MEK inhibitors, PI3K inhibitors, neprilysin inhibitors, beta2-agonists, CRTH2 antagonists, FXR agonists, BACE inhibitors, sphingosine-1-phosphate receptor modulators, MAPK inhibitors, Hedgehog signaling inhibitors, MDM2 antagonists, LSD1 inhibitors, lactamase inhibitors, TLR agonists, TLR antagonists, IDO inhibitors, ERK inhibitors, Chk1 inhibitors, splicing modulatory, DNA or RNA intercalators, etc. Other non-limiting examples of pharmacological agents as per the present invention includes for instance everolimus, trabectedin, abraxane, pazopanib, enzastaurin, vandetanib, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, pemetrexed, erlotinib, dasatanib, nilotinib, decatanib, panitumumab, amrubicin, oregovomab, nolatrexed, batabulin, ofatumumab, zanolimumab, edotecarin, tetrandrine, rubitecan, tesmilifene, oblimersen, ticilimumab, ipilimumab, gossypol, cilengitide, gimatecan, lucanthone, neuradiab, vitespan, talampanel, atrasentan, romidepsin, sunitinib, 5-fluorouracil, vorinostat, etoposide, gemcitabine, doxorubicin, 5′-deoxy-5-fluorouridine, vincristine, temozolomide, seliciclib, capecitabine, camptothecin, PEG-labeled irinotecan, tamoxifen, toremifene citrate, anastrazole, exemestane, letrozole, vatalanib, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, erlotinib, lapatanib, canertinib, lonafarnib, tipifarnib, amifostine, suberoyl analide hydroxamic acid, valproic acid, trichostatin sorafenib, arnsacrine, anagrelide, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, squalamine, endostatin, vitaxin, droloxifene, idoxyfene, spironolactone, finasteride, cimitidine, trastuzumab, denileukin diftitox,gefitinib, bortezimib, paclitaxel, cremophor-free paclitaxel, docetaxel, epithilone B, droloxifene, 4-hydroxytamoxifen, pipendoxifene, arzoxifene, fulvestrant, acolbifene, lasofoxifene, idoxifene, topotecan, rapamycin, temsirolimus, zolendronate, prednisone, lenalidomide, gemtuzumab, hydrocortisone, dexrazoxane, alemtuzumab, all-transretinoic acid, ketoconazole, megestrol, immune globulin, nitrogen mustard,methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa, efavirinz among others. Furthermore, as abovementioned, the pharmacological agents as per the present invention also includes naturally derived compounds which may for instance be obtained via purification from natural sources, any type of nucleic acid-based compounds, for instance oligonucleotides such as siRNA, splice-switching RNA, CRISPR guide strands, short hairpin RNA, antisense oligonucleotides, mRNA, and in particular nucleic acid-based agents which are chemically synthesized and/or which comprise chemically modified nucleotides such as 2′-O-Me, 2′-O-Allyl, 2′-O-MOE, 2′-F, 2′-CE, 2′-EA 2′-FANA, LNA, CLNA, ENA, PNA, phosphorothioates, tricyclo-DNA, etc. Furthermore, peptides and polypeptides, and not only peptides and/or proteins obtainable via peptide synthesis but also peptides and proteins obtainable through recombinant protein production, are also comprised within the definition of the pharmacological agents as per the present invention. As mentioned above, the present invention is naturally applicable also to other pharmacological agents without departing from the gist of the invention, as would be clear to a person skilled in the art.

In yet another embodiment, the EVs as per the present invention may comprise at least one targeting moiety displayed on the surface of the EV, to even further enhance its therapeutic potential by targeting a tissue, an organ, or cell type of interest. The targeting moiety normally comprises a sequence of amino acids, which may be identified for instance through phage display or any other type of screening methodology. The targeting moiety is typically displayed on the EV surface through genetic engineering of the EV source cells, wherein the source cells are transfected to produce EVs comprising a fusion protein comprising the targeting moiety and an exosome protein. Antibodies, such as monoclonal antibodies, can also function as targeting agents, especially when attached to EV surfaces.

In a further aspect, the present invention relates to a method of delivering a pharmacological agent to a target cell. Such delivery methods may comprise exposing a target cell, or a target tissue or target organ (which may include fluids and liquids such as blood, interstitial fluid, cerebrospinal fluid, etc.), to an EV as per the present invention. As above-mentioned, the EVs may comprise a targeting moiety expressed on its surface, or it may rely on natural tropism and targeting, or it may be non-targeted. Delivery to a target cell can be carried out in vitro and/or in vivo, depending on the context. Further, the present invention pertains to a method of altering the pharmacokinetic or pharmacodynamics profile of a small molecule drug. This can be achieved through loading the pharmacological agent in question into an EV, which will naturally affect factors such as distribution, enzymatic activity, tissue penetration, etc.

In a further aspect, the present invention relates to EVs comprising a pharmacological drug, wherein the pharmacological agent is released in the EV from a conjugate comprising the pharmacological agent and a metabolic component. Depending on the nature of the chemical link, a residue (such as a thiol, a biotin, a hydroxyl group, etc.) may remain on the small molecule agent after release from the metabolic component.

In yet another aspect, the present invention pertains to pharmaceutical compositions comprising EVs comprising metabolically loaded drugs. Typically, the pharmaceutical compositions as per the present invention comprise one type of therapeutic EV (i.e. a population of EVs comprising a certain desired pharmacological agent(s)) formulated with at least one pharmaceutically acceptable excipient, but more than one type of EV population may be comprised in a pharmaceutical composition, for instance in cases where a combinatorial treatment is desirable. The at least one pharmaceutically acceptable excipient may be selected from the group comprising any pharmaceutically acceptable material, composition or vehicle, for instance a solid or liquid filler, a diluent, an excipient, a carrier, a solvent or an encapsulating material, which may be involved in e.g. suspending, maintaining the activity of or carrying or transporting the EV population from one organ, or portion of the body, to another organ, or portion of the body (e.g. from the blood to any tissue and/or organ and/or body part of interest).

The present invention also relates to cosmetic and dermatological applications of EVs comprising pharmacological agents. Thus, the present invention pertains to skin care products such as creams, lotions, gels, emulsions, ointments, pastes, powders, liniments, sunscreens, shampoos, etc., comprising a suitable EV, in order to improve and/or alleviate symptoms and problems such as dry skin, wrinkles, folds, ridges, and/or skin creases. In one embodiment, EVs (which comprise a drug of interest) are obtained from a suitable EV-producing cell source with regenerative properties (for instance a mesenchymal stromal cell) are comprised in a cosmetic cream, lotion, or gel for use in the cosmetic or therapeutic alleviation of wrinkles, lines, folds, ridges and/or skin creases.

In yet another aspect, the present invention relates to EVs as per the present invention for use in medicine. Naturally, when an EV comprising a pharmacological agent in accordance with the present invention is used in medicine, it is in fact normally a population of EVs that is being used. The dose of EVs administered to a patient will depend on the amount of drug that has been loaded into the EV, the disease or the symptoms to be treated or alleviated, the administration route, the pharmacological action of the drug itself, the inherent properties of the EV, as well as various other parameters of relevance.

The EVs, the EV populations and/or the pharmaceutical compositions as per the present invention may thus be used for prophylactic and/or therapeutic purposes, e.g. for use in the prophylaxis and/or treatment and/or alleviation of various diseases and disorders. A non-limiting sample of diseases wherein the EVs as per the present invention may be applied comprises Crohn's disease, ulcerative colitis, ankylosing spondylitis, rheumatoid arthritis, multiple sclerosis, systemic lupus erythematosus, sarcoidosis, idiopathic pulmonary fibrosis, psoriasis, tumor necrosis factor (TNF) receptor-associated periodic syndrome (TRAPS), deficiency of the interleukin-1 receptor antagonist (DIRA), endometriosis, autoimmune hepatitis, scleroderma, myositis, stroke, acute spinal cord injury, vasculitis, non-alcoholic steatohepatitis (NASH), non-alcoholic fatty liver disease (NAFLD), fibrosis, Guillain-Barré syndrome, acute myocardial infarction, ARDS, sepsis, meningitis, encephalitis, liver failure, kidney failure, heart failure or any acute or chronic organ failure and the associated underlying etiology, graft-vs-host disease, Duchenne muscular dystrophy and other muscular dystrophies, lysosomal storage diseases such as Gaucher disease, Fabry's disease, MPS I, II (Hunter syndrome), and III, Niemann-Pick disease, Pompe disease, etc., neurodegenerative diseases including Alzheimer's disease, Parkinson's disease, Huntington's disease and other trinucleotide repeat-related diseases, dementia, ALS, cancer-induced cachexia, anorexia, diabetes mellitus type 2, and various cancers. Virtually all types of cancer are relevant disease targets for the present invention, for instance, Acute lymphoblastic leukemia (ALL), Acute myeloid leukemia, Adrenocortical carcinoma, AIDS-related cancers, AIDS-related lymphoma, Anal cancer, Appendix cancer, Astrocytoma, cerebellar or cerebral, Basal-cell carcinoma, Bile duct cancer, Bladder cancer, Bone tumor, Brainstem glioma, Brain cancer, Brain tumor (cerebellar astrocytoma, cerebral astrocytoma/malignant glioma, ependymoma, medulloblastoma, supratentorial primitive neuroectodermal tumors, visual pathway and hypothalamic glioma), Breast cancer, Bronchial adenomas/carcinoids, Burkitt's lymphoma, Carcinoid tumor (childhood, gastrointestinal), Carcinoma of unknown primary, Central nervous system lymphoma, Cerebellar astrocytoma/Malignant glioma, Cervical cancer, Chronic lymphocytic leukemia, Chronic myelogenous leukemia, Chronic myeloproliferative disorders, Colon Cancer, Cutaneous T-cell lymphoma, Desmoplastic small round cell tumor, Endometrial cancer, Ependymoma, Esophageal cancer, Extracranial germ cell tumor, Extragonadal Germ cell tumor, Extrahepatic bile duct cancer, Eye Cancer (Intraocular melanoma, Retinoblastoma), Gallbladder cancer, Gastric (Stomach) cancer, Gastrointestinal Carcinoid Tumor, Gastrointestinal stromal tumor (GIST), Germ cell tumor (extracranial, extragonadal, or ovarian), Gestational trophoblastic tumor, Glioma (glioma of the brain stem, Cerebral Astrocytoma, Visual Pathway and Hypothalamic glioma), Gastric carcinoid, Hairy cell leukemia, Head and neck cancer, Heart cancer, Hepatocellular (liver) cancer, Hodgkin lymphoma, Hypopharyngeal cancer, Intraocular Melanoma, Islet Cell Carcinoma (Endocrine Pancreas), Kaposi sarcoma, Kidney cancer (renal cell cancer), Laryngeal Cancer, Leukemias ((acute lymphoblastic (also called acute lymphocytic leukemia), acute myeloid (also called acute myelogenous leukemia), chronic lymphocytic (also called chronic lymphocytic leukemia), chronic myelogenous (also called chronic myeloid leukemia), hairy cell leukemia)), Lip and Oral, Cavity Cancer, Liposarcoma, Liver Cancer (Primary), Lung Cancer (Non-Small Cell, Small Cell), Lymphomas, AIDS-related lymphoma, Burkitt lymphoma, cutaneous T-Cell lymphoma, Hodgkin lymphoma, Non-Hodgkin, Medulloblastoma, Merkel Cell Carcinoma, Mesothelioma, Metastatic Squamous Neck Cancer with Occult Primary, Mouth Cancer, Multiple Endocrine Neoplasia Syndrome, Multiple Myeloma/Plasma Cell Neoplasm, Mycosis Fungoides, Myelodysplastic/Myeloproliferative Diseases, Myelogenous Leukemia, Chronic Myeloid Leukemia (Acute, Chronic), Myeloma, Nasal cavity and paranasal sinus cancer, Nasopharyngeal carcinoma, Neuroblastoma, Oral Cancer, Oropharyngeal cancer, Osteosarcoma/malignant fibrous histiocytoma of bone, Ovarian cancer, Ovarian epithelial cancer (Surface epithelial-stromal tumor), Ovarian germ cell tumor, Ovarian low malignant potential tumor, Pancreatic cancer, Pancreatic islet cell cancer, Parathyroid cancer, Penile cancer, Pharyngeal cancer, Pheochromocytoma, Pineal astrocytoma, Pineal germinoma, Pineoblastoma and supratentorial primitive neuroectodermal tumors, Pituitary adenoma, Pleuropulmonary blastoma, Prostate cancer, Rectal cancer, Renal cell carcinoma (kidney cancer), Retinoblastoma, Rhabdomyosarcoma, Salivary gland cancer, Sarcoma (Ewing family of tumors sarcoma, Kaposi sarcoma, soft tissue sarcoma, uterine sarcoma), Sézary syndrome, Skin cancer (nonmelanoma, melanoma), Small intestine cancer, Squamous cell, Squamous neck cancer, Stomach cancer, Supratentorial primitive neuroectodermal tumor, Testicular cancer, Throat cancer, Thymoma and Thymic carcinoma, Thyroid cancer, Transitional cell cancer of the renal pelvis and ureter, Urethral cancer, Uterine cancer, Uterine sarcoma, Vaginal cancer, Vulvar cancer, Waldenström macroglobulinemia, and/or Wilm's tumor.

The drug-loaded EVs as per the present invention may be administered to a human or animal subject via various different administration routes, for instance auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the characteristics of the pharmacological agent and/or the EV population as such.

The methods of loading pharmacological agents into EVs described herein are highly efficient and easily scalable, and allow for the rapid production of drug-loaded EVs in quantities needed for therapeutic administration. In certain embodiments of the foregoing aspects, when loading of EVs directly with pharmacological agents the loading may occur in 30 minutes or less, e.g. 5 minutes or less. In some embodiments, loading of the EVs occurs in 30 minutes, 20 minutes, 15 minutes, 10 minutes, 5 minutes, or 1 minute. In certain embodiments, at least 80% of the EVs in a given EV population may be loaded with the drug in question. In a preferred embodiment, at least 90% of the EVs are loaded with the drug. In exemplary embodiments, at least 91%, at least 92%, at least 93%, at least 94%, at least 95%, at least 96%, at least 97%, at least 98% or more of the EVs are loaded with the drug. Naturally, when metabolically loading EV source cells then the loading of EVs may take longer, as the loading may be dependent on transport processes and/or metabolic processes inside of EV source cells.

The methods of the present invention may also comprise exposing the EV source cells to serum starvation, hypoxia, bafilomycin, or cytokines such as TNF-alpha and/or IFN-gamma, in order to influence the yield or properties of the resulting EVs. The EV production scale and timeline will be heavily dependent on the EV-producing cell or cell line and may thus be adapted accordingly by a person skilled in the art. The methods as per the present invention may further comprise an EV purification step, wherein the EVs are purified through a procedure selected from the group of techniques comprising liquid chromatography (LC), high-performance liquid chromatography (HPLC), spin filtration, tangential flow filtration, hollow fiber filtration, centrifugation, immunoprecipitation, flow field fractionation, dialysis, microfluidic-based separation, etc., or any combination thereof. Naturally, depending on whether the EVs are loaded with the drug via metabolic loading of EV source cells or loading directly of EVs, the purification of EVs may be adapted accordingly. Typically, when loading EVs directly the EV population to be loaded has already gone through at least one purification step. On the other hand, when loading EV source cells the purification step is applied to “pre-loaded” EVs released/secreted from the EV source cells. In an advantageous embodiment, the purification of the EVs is carried out using a sequential combination of filtration (preferably ultrafiltration (UF), tangential flow filtration or hollow fiber filtration) and size exclusion liquid chromatography (LC). This combination of purification steps results in optimized purification, which in turn leads to superior therapeutic activity. Further, as compared to ultracentrifugation (UC), which is routinely employed for purifying exosomes, sequential filtration-chromatography is considerably faster and possible to scale to higher manufacturing volumes, which is a significant drawback of the current UC methodology that dominates the prior art. Another advantageous purification methodology is tangential flow filtration (TFF), which offers scalability and purity, and may be combined with others types of purification techniques such as filtration. A typical workflow for the production of small molecule-carrying EVs comprises the steps of (1) creation of a stable cell line expressing EVs (optionally having a targeting moiety displayed on their surface), (2) in an optional step purification of large quantities of such (optionally genetically engineered) EVs, (3) introduction of at least one pharmacological agent into the EVs in question, through exposing the EV population to a conjugate comprising a pharmacological agent conjugated to a metabolic component. Another typical workflow for the production of drug-loaded EVs comprises the steps of (1) creation of a stable cell line expressing EVs (optionally having a targeting moiety displayed on their surface), (2) metabolically loading of EV source cells through culturing EV source cells in the presence of a conjugate comprising a pharmacological agent conjugated to a metabolic component, (3) purification of the drug-loaded EVs produced by the EV source cells.

It shall be understood that the above described exemplifying aspects, embodiments, alternatives, and variants can be modified without departing from the scope of the invention. The invention will now be further exemplified with the enclosed examples, which naturally also can be modified considerably without departing from the scope and the gist of the invention.

### EXAMPLES

### Example 1

### Loading of Immune Cell-Derived EVs with Cytosine Arabinoside via Fatty Acid Conjugation

Peripheral blood mononuclear cells (PBMCs) were extracted from whole blood and plated with an appropriate density in cell media. The cell media is removed after 24 hours and the plate is washed with PBS 3 times. New fresh EV-depleted media or serum free media was added, containing C18 fatty acid conjugated cytosine arabinoside (C18-AraC), to preload EV-producing cells with the drug for 1 h. Thereafter, cells were washed and media replaced with new fresh EV-depleted media or serum free media, and incubated 24 h to allow for the drug incorporation to the membrane of secreted EVs via endogenous metabolic pathways. EVs were purified from the conditioned media. Alternatively, EVs derived from untreated cells were isolated first, and then loaded with C18-AraC by co-incubation, and unloaded drug was eliminated by a wash step. The loading capacity of C18-AraC in secreted EVs was quantified by HPLC.

The media that the cells are grown in is normally depleted of foreign EVs and microparticles by ultracentrifugation at 110 000 g overnight before incubation with the cells. Alternatively, serum free media is applied in its place, such as OptiMEM or DMEM. Conditioned media from the PBMC culture is purified using different techniques, in this case ultrafiltration with sequential LC, or tangential flow filtration (TFF).

The cytotoxicity of free C18-AraC, EVs loaded with C18-AraC (via endogenous and exogenous metabolic pathway), and control EVs were determined using the MTT assay. MDA-MB-231 cells (1×10⁵ cells/100 μl/well) were cultured in 96-well plates at 37° C. and 5% CO₂. Equivalent C18-AraC concentrations of 0.1, 1, 10, 100 and 1000 nM. After an incubation time of 4 h, the MTT solution (2 mg/ml PBS) was added, the plates were incubated for 4 h, and the cells were lysed with 50% N,N-dimethylformamide containing 20% SDS, pH 4.5. The absorbance at 570 nm was measured for each well by the SpectraMax M5 instrument (Molecular Devices, CA).

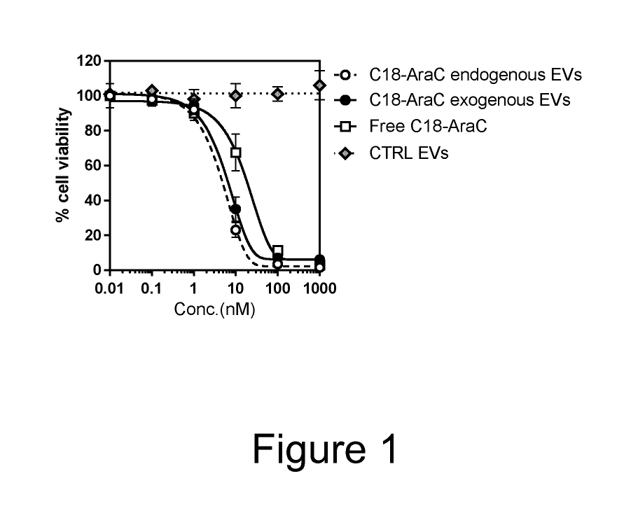

The absorbance of control cells was taken as 100% viability, and the values of the treated cells were calculated as a percentage of control. The results are shown in FIG. 1, indicating that EVs loaded with C18-AraC display similar anticancer effects as free C18-AraC, warranting further in vivo investigation. Using a similar experimental setup, an siRNA targeting cMyc was conjugated to C18 and loaded directly into EVs, either with or without electroporation at 100 V/cm. EVs loaded with the siRNA showed distinct antiproliferative effects, unlike a scrambled siRNA-C18 which had no effect on the cell viability.

### Example 2

### Loading of MSC-EVs with NSAID via Phospholipid Conjugation

Mesenchymal stem cells (MSCs) were plated with an appropriate density in cell media. The cell media was removed after 24 hours and the plate is washed with PBS 3 times. New fresh EV-depleted media or serum free media was added, containing a phospholipid conjugated to isobutylphenylpropanoic acid via its phosphor head group (PhLip-IBU), to preload EV-producing cells with the drug for 1 h. Thereafter, cells were washed and media replaced with new fresh EV-depleted media or serum free media, and incubated 24 h to allow for the drug incorporation to the membrane of secreted EVs via endogenous metabolic pathways. EVs were purified from the conditioned media (PhLip-IBU endogenous EVs). Alternatively, EVs derived from untreated cells were isolated first, and then loaded with PhLip-IBU by co-incubation, and unloaded drug was eliminated by a wash step (PhLip-IBU exogenous EVs). The loading capacity of PhLip-IBU in secreted EVs was quantified by HPLC.

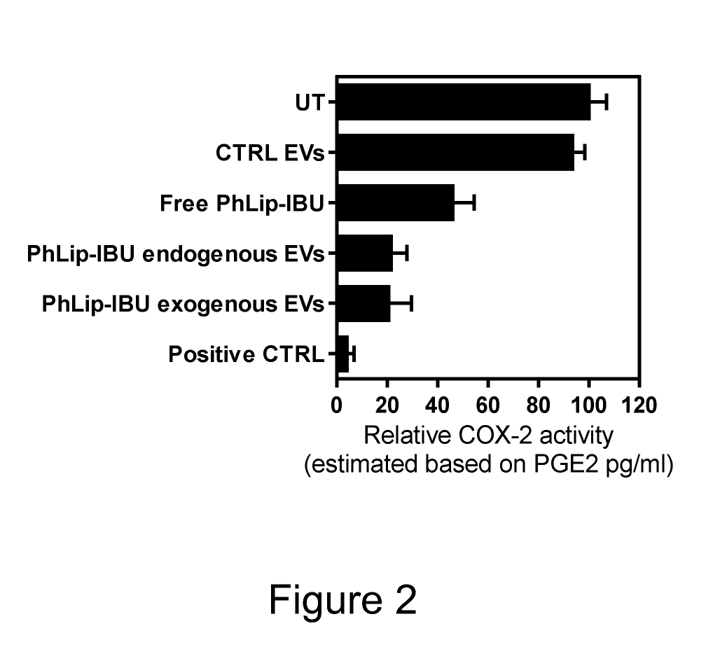

EVs were obtained and processed as in Example 1. The COX-2 inhibition of EV-loaded and free PhLip-IBU was measured in RAW264.7 cell, seeded in a 24-well plate. The following day, cells were pre-incubated with free PhLip-IBU, EV PhLIp-IBU or CTRL EVs, and then stimulated with 100 ng/ml LPS for 24 h. Cell culture supernatants were collected, centrifuged 15 min at 1000×g, and COX-2 inhibition assessed by measuring the activity of the downstream PGE2 using an appropriate ELISA kit. FIG. 2 indicates that COX-2 inhibition is enhanced by EVs, as compared to free PhLip-IBU.

### Example 3

### Loading of MSC-EVs with NSAID via Vitamin V7 or B9 Conjugation

MSCs stably expressing streptavidin, acceptor peptide, and/or folate receptor alpha (FRα) were cultured and prepared as in Example 2, but fed with heterotrimeric conjugates of isobutylphenylpropanoic acid and biotin (IBU-B7) or isobutylphenylpropanoic acid and folic acid (IBU-B9). Alternatively, isolated wild-type exosomes were incubated exogenously with IBU-B7 or IBU-B9. The loading capacity of IBU-B7 and IBU-B9 in secreted EVs was quantified by HPLC.

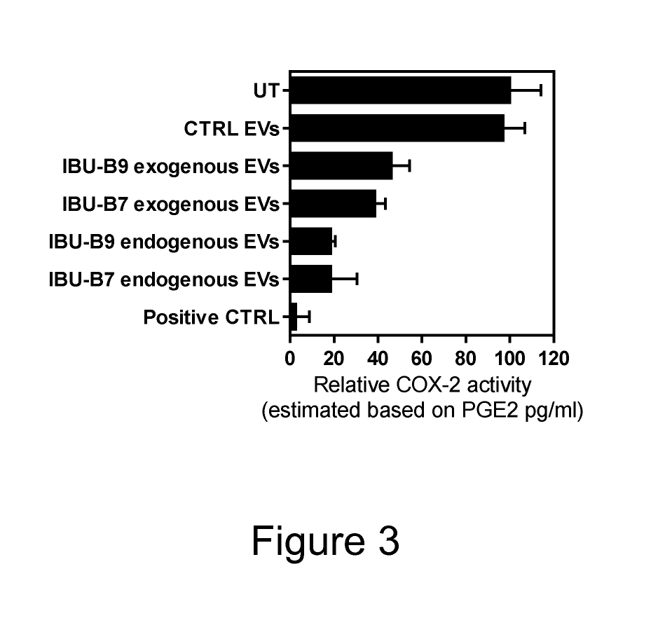

EVs were obtained and processed as in Example 1. The COX-2 inhibition of EV-loaded and free IBU-B7 and IBU-B9 was measured in RAW264.7 cell, seeded in a 24-well plate. The following day, cells were pre-incubated with free PhLip-IBU, EV PhLIp-IBU or CTRL EVs, and then stimulated with 100 ng/ml LPS for 24 h. Cell culture supernatants were collected, centrifuged 15 min at 1000×g, and COX-2 inhibition assessed by measuring the activity of the downstream PGE2 using an appropriate ELISA kit. FIG. 3 indicates that COX-2 inhibition achieved by MSC EVs loaded with IBU-B7 and IBU-B9.

1. A method for metabolically loading an extracellular vesicle (EV) with a pharmacological agent, comprising culturing EV source cells in the presence of a conjugate comprising the pharmacological agent conjugated to a metabolic component.

2. The method according to claim 1, wherein the conjugate comprises a chemical link between the pharmacological agent and the metabolic component.

3. The method according to claim 2, wherein the chemical link between the pharmacological agent and the metabolic component is a link that may dissociate, break or be cleaved to release the pharmacological agent.

4. The method according to any of the preceding claims, wherein the EV source cells are cultured under conditions which favor metabolic incorporation of the metabolic component.

5. The method according to claim 4, wherein the conditions which favor metabolic incorporation are low levels or essentially complete absence of unconjugated metabolic component, hypoxia, cytokine exposure, and/or other forms of cellular stress.

6. An EV obtainable by the methods of any one of the preceding claims.

7. An EV comprising a pharmacological agent conjugated to a metabolic component via a chemical link.

8. The EV according to claim 7, wherein the chemical link between the pharmacological agent and the metabolic component is a link that may dissociate, break or be cleaved to release the pharmacological agent.

9. The EV according to any one of claims 6 to 8, wherein the pharmacological agent is an anticancer agent, a cytostatic agent, a DNA or RNA intercalator, a splicing modulator, a tyrosine kinase inhibitor, a statin, an NSAID, an antibiotic, an antifungal agent, an antibacterial agent, an anti-inflammatory agent, an anti-fibrotic, an antihypertensive, an aromatase inhibitor, an esterase inhibitor, an anticholinergic, an SSRI, a BKT inhibitor, a PPAR agonist, a HER inhibitor, an AKT inhibitor, a BCR-ABL inhibitor, a signal transduction inhibitor, an angiogenesis inhibitor, a synthase inhibitor, an ALK inhibitor, a BRAF inhibitor, a MEK inhibitor, a PI3K inhibitor, a neprilysin inhibitor, a beta2-agonist, a CRTH2 antagonist, an FXR agonist, a BACE inhibitor, a sphingosine-1-phosphate receptor modulator, a MAPK inhibitor, an Hedgehog signaling inhibitor, an MDM2 antagonist, an LSD1 inhibitor, a lactamase inhibitor, a TLR agonist, a TLR antagonist, an IDO inhibitor, an ERK inhibitor, a Chk1 inhibitor, a nucleic acid-based agent such as an oligonucleotide, siRNA, shRNA, antisense oligonucleotide, splice-switching oligonucleotide, mRNA, a peptide, a natural product, a polypeptide, and any combination thereof.

10. The EV according to any one of claims 6 to 9, wherein the metabolic component is a lipid, a peptide or a protein, a mono-, di- or polysaccharide, a vitamin, a sterol, a ganglioside, an EV or cell membrane component, or any combination thereof.

11. The EV according to any one of claims 6 to 10, further comprising a targeting moiety.

12. The EV according to claim 11, wherein the targeting moiety comprises a sequence of amino acids expressed as a fusion protein with an EV polypeptide or an antibody attached to the surface of the EV.

13. An EV comprising a pharmacological agent, characterized in that the pharmacological agent is released in the EV from a conjugate comprising a metabolic component.

14. A pharmaceutical composition comprising a population of EVs according to any one of claims 6 to 13 and a pharmaceutically acceptable excipient.

15. EVs according to claims 6-13 and/or a pharmaceutical composition according to claim 14, for use in medicine.
